(19) United States
(12) Patent Application Publication
(10) Pub. No.: US 2020/0165350 A1
(43) Pub. Date: May 28, 2020

(54) STRONG POTENTIATION OF IMMUNE CHECKPOINT BLOCKADE THERAPY OF CANCER BY COMBINATION WITH ENDOGLIN-TARGETING THERAPY

(71) Applicant: Health Research, Inc., Buffalo, NY (US)

(72) Inventor: Ben K. SEON, Williamsville, NY (US)

(21) Appl. No.: 16/618,525

(22) PCT Filed: May 31, 2018
(86) PCT No.: PCT/US18/35409

§ 371 Date: Dec. 2, 2019

Related U.S. Application Data
(60) Provisional application No. 62/513,617, filed on Jun. 1, 2017.

(51) Int. Cl. C07K 16/28; C07K 16/30; A61P 35/04

(52) U.S. Cl. CPC C07K 16/2896; C07K 16/2827; A61K 2039/507; A61P 35/04; C07K 2317/76; C07K 16/30

(57) ABSTRACT

Provided are methods that involve administering to an individual in need thereof an immune checkpoint inhibitor and a monoclonal antibody (mAb) or antigen binding fragment thereof, wherein the mAb or the fragment thereof binds with specificity to human endoglin. For cancer patients, the administration results in inhibition of tumor growth and/or inhibition of metastasis, and/or prolongation of the life of the individual.

### CROSS REFERENCE TO RELATED APPLICATIONS

This application claims priority to U.S. provisional application No. 62/513,617, filed Jun. 1, 2017, the disclosure of which is incorporated herein by reference.

### FIELD OF THE INVENTION

The present invention is related to the field of therapy of cancer and angiogenesis-associated diseases, and more specifically to compositions and methods for use in anticancer and anti-angiogenesis therapies.

### BACKGROUND OF THE INVENTION

There is urgent need for better compositions and methods that will improve the currently available compositions and methods for treating patients with advanced cancer and advanced angiogenesis-associated diseases, including but not necessarily limited to age-related macular degeneration (AMD). In many solid cancers and other angiogenesis-associated diseases, expression of endoglin on endothelial cells is increased compared with normal tissues.

Endoglin

Endoglin is a homodimeric tumor-associated cell membrane antigen with molecular size of 160 kilodaltons (kD) or 170 kD that is mainly expressed on leukemia cells and endothelial cells (Haruta and Seon, 1986, Proc. Natl. Acad. Sci. USA 83: 7898-7902; Gougos and Letarte, 1988, J. Immunol. 141: 1925-1933; Seon et al., 1997, Clin. Cancer Res. 3: 1031-1044). Endoglin's expression on endothelial cells is upregulated on proliferating endothelial cells of the tumor-associated vascular and lymphatic endothelium (Seon et al., Supra; Burrows et al., 1995, Clin. Cancer Res. 1: 1623-1634; Matsuno et al., 1999, Clin Cancer Res. 5: 371-382; Clasper et al., 2008, 68: 7293-7303). Endoglin is essential for angiogenesis/vascular development (Li et al., 1999, Science 284: 1534-1537; Arthur et al., 2000, Dev. Biol. 217: 42-53) and a co-receptor of TGF-β (Cheifetz et al., 1992, J. Biol. Chem. 267: 19027-19030).

Murine endoglin has been characterized as a dimer with molecular size of approximately 180 kilodaltons (kD). Human endoglin exists in two forms; i.e., a smaller 160 kD form and a larger 170 kD form with the difference between the two being found in the cytoplasmic portion of the protein. Endoglin has an extracellular region, a hydrophobic transmembrane region, and a cytoplasmic tail. A comparison of the nucleotide sequence of human endoglin with murine endoglin reveals an identity of about 71 to 72% (St. Jacques et al., 1994, Endocrinol. 134:2645-2657; Ge et al., 1994, Gene 158:2645-2657). However, in the human and murine sequences encoding the transmembrane regions and cyto-plasmic regions of endoglin, there is a 93-95% identity. Thus, in the human and murine sequences encoding the extracellular region to which antibody would be directed at the cell surface, there is significantly less identity than 70%. Although the amino acid sequence similarity between human and mouse endoglin appears substantial, the observed amino acid sequence differences in the extracellular regions should be sufficient for generating distinct antigenic epitopes unique to human endoglin or to mouse endoglin. This is because in peptide epitopes, even a subtle variation in the amino acid sequence comprising the epitopes or in the flanking amino acid sequences can markedly influence the immunogenicity of the epitopes (see, e.g., Vijayakrishnan et al., 1997, J. Immunol. 159:1809-1819). For instance, single amino acid substitutions in a peptide can cause marked changes in the immunogenicity of the peptide (Vijayakrishnan et al., 1997, supra). Such changes in a peptide epitope will strongly influence the specificity of mAbs because mAbs define fine specificity.

SN6 is an anti-endoglin monoclonal antibody generated from immunization of mice with tumor-associated components of glycoprotein mixtures of cell membranes of human leukemia cells (Haruta and Seon, 1986, Proc. Natl. Acad. Sci. 83:7898-7902). It is a murine mAb that recognizes human endoglin. mAb 44G4 is an antibody generated from immunization of mice with whole cell suspensions of human pre-B leukemia cells (Gougos and Letarte, 1988, J. Immunol. 141:1925-1933; 1990, J. Biol. Chern. 265:8361-8364). It is a murine mAb that recognizes human endoglin. mAb MJ7/18 is an antibody generated from immunization of rats with inflamed mouse skins (Ge and Butcher, 1994, supra). It is a mAb that recognizes murine endoglin. mAb Tec-11 is an antibody generated from immunization of mice with human umbilical vein endothelial cells (Burrows et al., 1995, Clin. Cancer Res. 1:1623-1634). It is a murine mAb with reactivity restricted to human endoglin.

By the use of anti-endoglin antibodies and various staining procedures known in the art, it has been determined that endoglin is expressed at moderate levels on human tumor cells such as from human leukemia, including non-T-cell-type (non-T) acute lymphoblastic leukemia (ALL), myelo-monocytic leukemia. In addition, it has been determined that endoglin is expressed at moderate to high levels in endothelial cells contained in tumor-associated vasculatures from human solid tumors, including angiosarcoma, breast carcinoma, cecum carcinoma, colon carcinoma, lung carcinoma, melanoma, osteo-sarcoma, ovarian carcinoma, parotid tumor, pharyngeal carcinoma, rectosigmoid carcinoma; and human vasculature from placenta, adrenal and lymphoid tissues. A lesser degree (weak) endothelial cell staining for endoglin has been observed in a variety of normal human adult tissue sections from spleen, thymus, kidney, lung and liver.

Increased endoglin expression on vascular endothelial cells has also been reported in pathological conditions involving angiogenesis. Such angiogenesis-associated diseases include most types of human solid tumors, rheumatoid arthritis, stomach ulcers, and chronic inflammatory skin lesions (e.g., psoriasis, dermatitis; Westphal et al., 1993, J. Invest. Dermatol. 100:27-34).

Angiogenesis

Angiogenesis is the formation of new capillary blood vessels leading to neovascularization. Angiogenesis is a complex process which includes a series of sequential steps including endothelial cell-mediated degradation of vascular basement membrane and interstitial matrices, migration of endothelial cells, proliferation of endothelial cells, and formation of capillary loops by endothelial cells. Solid tumors are angiogenesis-dependent; i.e., as a small solid tumor reaches a critical diameter, for further growth it needs to elicit an angiogenic response in the surrounding normal tissue. The resultant neovascularization of the tumor is associated with more rapid growth, and local invasion. Further, an increase in angiogenesis is associated with an increased risk of metastasis. Therefore, antiangiogenic therapy to inhibit tumor angiogenesis would suppress or arrest tumor growth and its spread.

In normal physiological processes such as wound healing, angiogenesis is turned off once the process is completed. In contrast, tumor angiogenesis is not self-limiting. Further, in certain pathological (and nonmalignant) processes, angiogenesis is abnormally prolonged. Such angiogenesis-associated diseases include diabetic retinopathy, chronic inflammatory diseases including rheumatoid arthritis, dermatitis, and psoriasis. Antiangiogenic therapy would allow modulation in such angiogenesis-associated diseases having excessive vascularization.

Antiangiogenic Therapy and Vascular Targeting Therapy of Solid Tumors

The progressive growth of solid tumors beyond clinically occult sizes (e.g., a few mm³) requires the continuous formation of new blood vessels, a process known as tumor angiogenesis. Tumor growth and metastasis are angiogenesis-dependent. A tumor must continuously stimulate the growth of new capillary blood vessels to deliver nutrients and oxygen for the tumor itself to grow. Therefore, either prevention of tumor angiogenesis (antiangiogenic therapy) or selective destruction of tumor's existing blood vessels (vascular targeting therapy) present a strategy directed to preventing or treating solid tumors.

Since a local network of new capillary blood vessels provide routes through which the primary tumor may metastasize to other parts of the body, antiangiogenic therapy should be important in preventing establishment of small solid tumors or in preventing metastasis (See, e.g., Folkman, 1995, Nature Medicine, 1:27-31). On the other hand, the vascular targeting therapy which attacks the existing vasculature is likely to be most effective on large tumors where the vasculature is already compromised (See, e.g., Bicknell and Harris, 1992, Semin. Cancer Biol. 3:399-407). Monoclonal antibodies, and fragments thereof according to the present invention are used as a means of delivering to either existing tumor vasculature or newly forming tumor neovascularization therapeutic compounds in a method of antiangiogenic therapy and vascular targeting therapy (collectively referred to as “antiangiogenic therapy”).

Mouse Models for Cancer and Angiogenesis-Associated Diseases

The use of mouse models of cancer angiogenesis has been accepted and validated as a model for the evaluation of therapeutic agents because the models have been shown to reflect the clinical parameters characteristic of the respective disease, as well as predictive of the effectiveness of therapeutic agents in patients. These mouse models include, but are not limited to: mouse model for cancer development and therapy (Martin et al., 1986, Cancer Res., 46: 2189-2192; Hara and Seon, 1987, Proc. Natl. Acad. Sci., USA, 84: 3390-3394; Yokota et al., 1990, Cancer Res., 50: 32-39); mouse model for retinal neovascularization (Pierce et al., 1995, Proc. Natl. Acad. Sci. USA 92:905-909); mouse models for rheumatoid arthritis (MRL-1pr/1pr mouse model, Folliard et al., 1992, Agents Actions 36:127-135; mev mouse, Kovarik et al., 1994, J. Autoimmun. 7:575-88); mouse models for angiogenesis (Majewski et al., 1994, Int. J. Cancer 57:81-85; Andrade et al., 1992, Int. J. Exp. Pathol., 73:503-13; Sunderkotter et al., 1991, Am. J. Pathol. 138:931-939); mouse model for dermatitis (Maguire et al., 1982, J. Invest. Dermatol. 79:147-152); and mouse model for psoriasis (Blandon et al., 1985, Arch. Dermatol. Res. 277:121-125; Nagano et al., 1990, Arch. Dermatol. Res. 282:459-462). Concerning mouse model for endoglin targeted therapy of cancer and angiogenesis-associated diseases, the majority of mouse anti-human endoglin monoclonal antibodies do not cross-react with mouse endoglin and mouse endothelial cells (Seon et al., 2011, Current Drug Delivery 8: 135-143). Therefore, the majority of the prior art anti-endoglin monoclonal antibodies, which are restricted to reactivity with human endoglin, cannot be used in the mouse model for the respective angiogenesis-associated disease to perform the studies necessary to evaluate the clinical efficacy, pharmacokinetics, and adverse side effects of antiangiogenic therapy in humans. Recently we developed a new animal model expressing human endoglin that can be targeted by anti-human endoglin monoclonal antibodies, i.e., a genetically engineered mouse model (GEMM) expressing humanized endoglin (Toi et al., 2015, Int. J. Cancer, 136: 452-461). This animal model can be used to model antiangiogenic therapy of human tumors, and of other human angiogenesis-associated diseases having excessive vascularization, which can be evaluated for clinical efficacy and pharmacokinetics in mouse models of angiogenesis-associated diseases. In addition, this animal model is immunocompetent and maintains T cell functions. Therefore, this animal model can be used for evaluation of immune checkpoint blockade antibodies in cancer therapy.

Immune Checkpoint Blockade Therapy of Cancer

Development of the immune checkpoint blocking antibodies as cancer therapies is based on the natural role of the checkpoint molecules as coinhibitory receptors of T-cell activation (Sharma and Allison, 2015, Cell, 161: 205-214; Boussiotis, 2016, New Engl. J. Med. 375: 1767-1778). Strategies to unleash T cells against tumors are attractive for their specificity, memory and adaptability for cancer therapy (Sharma and Allison, Supra). Recently some immune checkpoint blocking antibodies including anti-CTLA-4 (cytotoxic T-lymphocyte-associated antigen 4), anti-PD-1 (programmed cell death protein-1) and anti-PD-L1 (programmed cell death-ligand 1) antibodies have been successful in treating subsets of patients with metastatic solid tumors such as melanoma, renal cell carcinoma, ovarian cancer and non-small cell lung cancer (Sharma and Allison, Supra; Robert et al., 2011, New Engl. J. Med. 2011, 364:2517-2526; Brahma et al., 2012, New Engl. J. Med. 366:2455-2465; Hamid et al., 2013, New Engl. J. Med. 369:134-144; Prieto et al., 2012, Clin. Cancer Res. 18:2039-2047; Larkin et al., 2015, New Engl. J. Med. 373:23-34). The therapies have been shown to be effective for subsets of cancer patients, and induced durable antitumor response in some patients. Nevertheless, many patients do not have a good response to monotherapy approaches with immune checkpoint blockers, and alternative strategies such as combination of immune checkpoint blockers with other types of anticancer agents are required to achieve significant therapeutic benefit these patients (Smyth et al., 2016, Nat. Rev. Clin. Oncol. 13:143-158).

Many clinical trials by a number of investigators indicated that anti-PD-1 mAb and anti-PD-L1 mAb may be the most effective anticancer agents among the known immune checkpoint blockade agents (e.g., Topalian et al., 2012, New Engl. J. Med. 366:2443-2454; Brahma et al., Supra; Moreno and Ribas, 2015, British J. Cancer 112:1421-1427; Boussiotis, 2016, New Engl. J. Med. 375:1767-1778). These clinical trials indicate that anti-PD-1 mAb and anti-PD-L1 mAb may be superior to anti-CTLA-4 mAb with regard to the prolonged progression-free survival and less high-grade toxicity (Robert et al., 2015, New Engl. J. Med. 372:2521-2532. However, there remains on ongoing and unmet need to improve the efficacy of checkpoint inhibitors. The present invention is pertinent to this and other needs.

### SUMMARY

The present disclosure provides compositions and methods that relate to prophylaxis and therapy of cancer and angiogenesis-associated diseases. The disclosure includes providing anti-human endoglin mAbs that strongly potentiate antitumor activity of immune checkpoint blocking antibody when they are combined with an immune checkpoint blocking antibody. The combination also potentiates antitumor activities of the anti-endoglin mAbs. The combination therapy with anti-endoglin mAbs and immune checkpoint blocking antibody is expected to be applicable to patients with a variety of cancers and angiogenesis-associated diseases, and is expected to exert a positive impact on the therapy of patients with advanced cancer and angiogenesis-associated diseases.

In general, methods of this disclosure comprise administering to an individual thereof a composition comprising one or more of anti-endoglin mAbs and an immune checkpoint blocking mAb or immunoconjugates developed from the mAbs. For each therapeutic use, the invention includes combination therapies using anti-endoglin mAbs and an immune checkpoint blocking mAb, immunoconjugates of the mAbs and other therapeutic agents.

In more detail, in the current disclosure we analyzed whether the combination of endoglin-targeted therapy and immune checkpoint blockade could exert additive or synergistic antitumor efficacy, and determined if the toxicity of the combination would be increased relative to known toxicities of each component of the combination. In a non-limiting demonstration, we selected anti-PD-L1 mAb as the immune checkpoint blockade agent to demonstrate certain features of this disclosure. Anti-PD-L1 mAb is designed to inhibit the interaction of PD-L1 with PD-1 and B7.1, relieving the inhibition of T-cell activity and allowing for antitumor immune response.

Without intending to be bound by any particular theory, it is considered that the mechanisms of endoglin-targeted therapy are distinct from those of immune checkpoint blockade. Mechanisms of endoglin-targeted therapy involve anti-angiogenesis, apoptosis induction of microvascular endothelial cells, and modulation of TGF-beta-mediated and bone morphogenetic protein 9 (BMP9)-mediated signal transduction (She et al., 2004, Int. J. Cancer, 108: 251-257; Tsujie et al., 2008, Int. J. Cancer, 122: 2266-2273; Uneda et al., 2009, Int. J. Cancer, 125: 1446-1453; Seon et al., 2011, Current Drug Delivery, 8: 135-143; Nolan-Stevaux et al., 2012, PLoS One, 7: e50920), whereas mechanisms of immune checkpoint blockade therapy involve blocking of the pathways inhibiting the endogenous immune response to cancer in the tumor-bearing host (Sharma and Allison, Supra). In addition, anti-ENG antibody TRC105 was recently found to significantly decrease the levels of regulatory T cells (Treg cells) in patients with advanced/metastatic cancers in clinical trials of TRC105 as a single agent (Karzai et al., 2015, BJU Int., 116: 546-555; Apolo et al., 2017, Clinical Geritourinary Cancer, 15: 77-85). TRC105 is a human/mouse chimeric antibody that was derived from mouse anti-human ENG mAb SN6j (Seon et al, 2011, Supra).

Adverse events of ENG-targeted therapy in cancer patients do not overlap with those of immune checkpoint blockade in cancer patients. Adverse events of anti-ENG mAb TRC105 when it was administered as a single agent into cancer patients included infusion-related reaction, anemia, fatigue, telangiectasia and nausea (Rosen et al., 2012, Clin. Cancer Res., 18: 4820-4829; Karzai et al., Supra; Apolo et al., Supra). Adverse events of anti-CTLA-4 mAb, anti-PD-1 mAb and anti-PD-L1 mAb included asthenia, rash, adverse cutaneous reactions, decreased appetite, diarrhea and nausea (Cousin and Italiano, 2016, Clin. Cancer Res., 22: 4550-4555; Simone et al., 2016, Clin. Cancer Res., 22: 4023-4029).

Combination Therapy of Cancer with Anti-Endoglin Antibodies and Immune Checkpoint Blocking Antibodies

Several clinical trials indicated that anti-PD-1 (programmed cell death protein-1) mAb and anti-PD-L1 (programmed cell death-ligand 1) mAb may be superior to anti-CTLA-4 (cytotoxic T-lymphocyte-associated antigen 4) mAb with regard to prolonged progression-free survival and less high-grade toxicity (e.g., Robert et al., Supra). We analyzed anti-PD-L1 mAb as a representative checkpoint blocker in the present disclosure.

Anti-PD-L1 is designed to inhibit the interaction of PD-L1 with PD-1 and B7.1, relieving the inhibition on T-cell activity and allowing for antitumor immune response. In the initial experiment, we performed a dose-dependent titration experiment of an rat anti-mouse PD-L1 mAb (from 10F.9G2 clone, BioLegend) by intravenous (i.v.) administration of the mAb into GEMs bearing established tumors of 4T1 murine breast cancer cells at 3-day intervals to investigate an optimal dose for antitumor efficacy and side effects. The anti-PD-L1 mAb showed a dose-dependent antitumor activity between 100 μg/mouse and 250 μg/mouse and no significant toxic effects were observed. The disclosure is therefore expected to be appropriate for use in a variety of anti-cancer approaches and can been evaluated by measuring a variety of parameters that include tumor size, metastatic nodules in the distant organs, microvessel density in the tumors, survival of the treated mice and body weight (Takahashi et al, 2001, Cancer Res., 61: 7846-7854; Tsujie et al, Supra, Uneda et al., Supra; Toi et al., 2015, Int. J. Cancer, 136: 452-461; Seon et al., 2011, Supra).

### BRIEF DESCRIPTION OF FIGURES

FIG. 1 depicts suppression of growth of 4T1 breast tumor in genetically engineered mice (GEMs) expressing humanized endoglin by systemic administration of anti-PD-L1 monoclonal antibody (mAb) (an immune checkpoint blocking mAb), endoglin-targeting mAb SN6j or SN6h, a control mouse IgG CCL130, or combination of anti-PD-L1 mAb with anti-endoglin mAb. 4T1 murine breast cancer cells were inoculated subcutaneously (s.c.) into mice and mice with a similar size of tumor were distributed nearly evenly into 6 groups of mice (n=7 for each group) at the onset of the therapy. 4T1 breast tumor spontaneously metastasizes in distant organs including lung. The anti-mouse PD-L1 mAb 10F.9G2 (from BioLegend) was given intravenously (i.v.) at the dose of 300 μg/mouse and at 3-day intervals while anti-human endoglin mAb SN6j or SN6h and control mouse IgG CCL130 were given i.v. individually at the dose of 200 μg/mouse and at 2-day intervals. The last injection of the anti-PD-L1 mAb is indicated by an arrow on the abscissa while the last injection of SN6j, SN6h or CCL130 is indicated by an arrow head on the abscissa. Mice were followed daily, and tumor size and body weight of mice were measured at 2 or 3 day intervals as described previously (Takahashi et al., 2001, Supra). The measured tumor diameters were converted to tumor volume using the Excel program and the following formula: V=length×width×height×π/6 (Toi et al, 2015, Supra). Statistically significant differences between different groups in the tumor size are indicated by star symbols (* : <0.05; ** : <0.01; *** : <0.001; **** : <0.0001). Each tumor growth curve represents the average of tumor sizes of 7 mice in each group and the standard deviation is indicated in individual curves. Anti-PD-L1 mAb shows a weak but significant tumor suppressive activity compared with the control, CCL130 (p<0.05). Both SN6j and SN6h strongly suppressed tumor growth compared with the control CCL130 (p<0.001 and <0.0001, respectively). Addition of anti-PD-L1 mAb to SN6j or SN6h strongly potentiates antitumor activity compared with anti-PD-L1 mAb+CCL130 (p<0.01 for anti-PD-L1 mAb+SN6j and p<0.001 for anti-PD-L1 mAb+SN6h). In addition, the addition of anti-PD-L1 mAb potentiates antitumor activity compared with SN6j alone (p<0.05) and SN6h alone (p<0.05). SN6h is more effective than SN6j for tumor suppression (p<0.01).

FIG. 2 depicts metastasis suppression in the lungs of GEMs bearing 4T1 breast tumor by i.v. administration of anti-PD-L1 mAb and anti-endoglin mAb. 4T1 cells were inoculated s.c. in the flank of mice and mice bearing established 4T1 tumor was treated as described in the legend to FIG. 1. Mice were sacrificed 7 days after the last treatment with anti-endoglin mAb or control IgG and metastatic nodules in the lungs were counted. Addition of anti-endoglin mAb SN6j or SN6h to an immune checkpoint blocking anti-PD-L1 mAb potentiates metastasis suppressive activity compared with anti-PD-L1 mAb+CCL130 (p=0.0000099 for SN6j and p=0.0000015 for SN6h). It is remarkable that metastasis is completely suppressed in the lungs of 3 of 7 mice that received SN6h plus anti-PD-L1 mAb. SN6j and SN6h are also effective for suppressing metastasis compared with control IgG CCL130 (p=0.000067 for SN6j and p=0.0000032 for SN6h).

FIG. 3 depicts suppression of growth of Colon 26 colorectal tumor in mice by an immune checkpoint blocking anti-PD-L1 mAb and anti-endoglin (ENG) mAb SN6j and SN6h. Colon 26 tumor cells were subcutaneously (s.c.) injected into GEMs expressing humanized endoglin. Mice with a similar size of tumors were distributed nearly evenly into 7 groups (n=8 for each of 5 therapy groups and n=7 for each of 2 control groups) at the onset of the therapy. Individual groups of mice with established tumors were treated by intravenous (i.v.) administration of an isotype-matched control mouse IgG CCL130 (IgG1), an isotype-matched control rat IgG (IgG2b from BioLegend), rat anti-mouse PD-L1 mAb (IgG2b from clone 10F.9G2, BioLegend), mouse anti-human ENG mAb SN6j (IgG1) and SN6h (IgG1). The remaining two groups of mice were treated by i.v. administration of SN6j+anti-PD-L1 mAb and SN6h+anti-PD-L1 mAb, respectively. CCL130, SN6j and SN6h were individually administered at the dose of 150 μg/mouse and at 3-day intervals, whereas rat control IgG2b and anti-PD-L1 mAb were individually given at the dose of 300 μg/mouse and at 3-day intervals. The mice were followed daily, and tumor size and body weight were measured at 2 or 3 day intervals. Rat control IgG2b suppresses tumor growth significantly in the tumor-bearing mice compared with CCL130 mouse control IgG (p<0.01). Rat anti-mouse PD-L1 mAb shows significant tumor suppressive activity compared with the isotype-matched control rat IgG2b (p<0.001). SN6j and SN6h show stronger antitumor activity (p<0.0001, respectively) compared with CCL130. Combination of the anti-PD-L1 mAb with either SN6j or SN6h potentiates antitumor activity compared with either anti-PD-L1 mAb (p<0.001 and p<0.0001, respectively, for SN6j and SN6h) or with anti-ENG mAb (p<0.01 and p<0.001, respectively, for SN6j and SN6h). SN6h+anti-PD-L1 mAb is more potent than SN6j+anti-PD-L1 in the tumor growth suppression (p<0.001). Statistically significant differences between different groups in the tumor suppression are indicated by star symbols (* : <0.05; ** : <0.01; *** : <0.001; **** : <0.0001).

FIG. 4 depicts prolongation of survival of tumor-bearing mice by systemic administration of endoglin (ENG)-targeting antibody or combination of an immune checkpoint blockade antibody with an ENG-targeting antibody. Colon 26 colorectal tumor cells were inoculated s.c. into GEMs expressing humanized ENG and 7 groups of the tumor-bearing mice (n=8 for each of 5 therapy groups and n=7 for each of two control groups) were treated as described in FIG. 3. In the present test, survival of each group of mice was monitored and survival of relevant pairs of groups of mice is presented in FIG. 4, panel a) to panel h). FIG. 4, panel a) Anti-PD-L1 mAb shows a weak trend to extension of survival of tumor-bearing mice compared with the isotype-matched control rat IgG (IgG2b), but the extension is not statistically significant (p=0.163). FIG. 4, panel b) Anti-ENG mAb SN6j is effective for extending the survival of the tumor bearing mice compared with the isotype-matched control IgG1 CCL130 (p=0.0182). FIG. 4, panel c) Another anti-ENG mAb SN6h is also effective for extending the survival of the tumor-bearing mice compared with CCL130 (p=0.0240). FIG. 4, panel d) SN6j+anti-PD-L1 mAb is effective for prolonging the survival of tumor-bearing mice compared with anti-PD-L1 mAb alone (p=0.0353). FIG. 4, panel e) SN6h+anti-PD-L1 mAb is highly effective for prolonging the survival of the tumor-bearing mice compared with anti-PD-L1 mAb alone (p=0.0033). FIG. 4, panel f) Addition of anti-PD-L1 mAb to SN6j is not sufficiently effective for extending survival compared with SN6j alone (p=0.0847). FIG. 4, panel g) Addition of anti-PD-L1 mAb to SN6h strongly extended the survival of the tumor-bearing mice compared with SN6h alone (p=0.0078). FIG. 4, panel h) SN6h+anti-PD-L1 mAb appears to be more effective than SN6j+anti-PD-L1 mAb in treating the tumor-bearing mice but the difference in the survival extension between the two groups is not statistically significant (p=0.147). Nevertheless, SN6h+anti-PD-L1 mAb is more effective for survival prolongation of the tumor-bearing mice than either of anti-PD-L1 mAb alone (p=0.0033) and SN6h alone (p=0.0078). The statistical analysis of the survival was performed by use of Log-rank (Mantel-Cox) test and Gehan-Breslow-Wilcoxon test. The p values obtained by the two tests are similar. The presented p values are those that were obtained by the Log-rank (Mantel-Cox) test. We observe synergistic effect on the survival extension by the combination of anti-PD-L1 mAb with anti-ENG mAb SN6h compared with individual components of the combination as described below. In FIG. 4, panel a, treatment of tumor-bearing mice with anti-PD-L1 mAb extended the median survival (survival of the 50% of the mice) of the eight mice in the group by 2 days to 21 days compared with 19 days of the median survival for the control group that was treated with an isotype-matched control rat IgG2b. In FIG. 4, panel c, treatment of the eight tumor-bearing mice with SN6h extended the median survival by 2 days to 21 days compared with 19 days of the median survival of the control group that was treated with an isotype-matched control mouse IgG1 CCL130. In FIG. 4, panel e, treatment of eight tumor-bearing mice with the combination of SN6h plus anti-PD-L1 mAb extended the median survival by 9 days to 30 days compared with 21 days of the median survival of the group of the eight tumor-bearing mice that were treated with anti-PD-L1 mAb. This survival extension of 9 days is equivalent to extension of 11 days compared with two control groups of rat IgG2b and mouse IgG1 CCL130. This extension of 11 days is much larger than the additive effect of 4 days (2 days+2 days) by SN6h and anti-PD-L1 mAb. The results show synergistic effect of the combination therapy with SN6h and anti-PD-L1 mAb.

FIG. 5 depicts tumor growth suppression in experiments in which growth of established 4T1 breast tumor in GEMs expressing humanized endoglin (ENG) is suppressed by systemic administration of murine control IgG CCL130 (IgG1), rat control IgG (IgG2b), rat anti-mouse PD-L1 monoclonal antibody (mAb), murine anti-human ENG mAb SN6j, murine anti-human ENG mAb SN6h, SN6j+anti-PD-L1 mAb, or SN6h+anti-PD-L1 mAb. CCL130 is an isotype-matched control IgG of SN6j and SN6h while rat IgG2b is an isotype-matched control IgG of the anti-PD-L1 mAb. Anti-PD-L1 mAb is an immune checkpoint blocking antibody. SN6j and SN6h define mutually non-overlapping distinctively different epitope of ENG (She et al., 2004, Supra). 4T1 murine breast tumor cells were inoculated subcutaneously (s.c.) into GEMs and mice with a similar size of established tumor were distributed nearly evenly into 7 groups of mice (n=10 for each group) at the onset of the therapy. The rat anti-mouse PD-L1 mAb 10F.9G2 (from BioLegend) or the control rat IgG2b (from BioLegend) was given intravenously (i.v.) at the dose of 250 μg/mouse and at 3-day intervals. SN6j, SN6h or the control IgG CCL130 was given i.v. at the dose of 200 μg/mouse at 2-day intervals. The last injection of anti-PD-L1 mAb or rat IgG2b is indicated by an arrow on the abscissa while the last injection of CCL130, SN6j or SN6h is indicated by an arrow head. Statistically significant differences between different groups in the therapeutic efficacy are indicated by star symbols (* :P<0.05; ** : P<0.01; *** : P<0.001; **** : P<0.0001). Mice were followed daily, and tumor size and body weight were measured at 2 or 3 days intervals. Rat IgG2b shows significant activity against tumor growth compared with CCL130, the mouse control IgG (P<0.001); this activity of rat IgG2b in mice is believed to be caused by side effects of the immune response of mice against hetero-species rat IgG. Rat anti-mouse PD-L1 mAb shows strong tumor-suppressive activity compared with the isotype-matched control rat IgG2b (P<0.0001). Both anti-ENG mAb SN6j and mAb SN6h show stronger tumor suppressive activities than anti-PD-L1 mAb (P<0.001 and <0.0001, respectively). Combination of SN6j or SN6h with anti-PD-L1 mAb strongly enhanced antitumor activity compared with individual components of the combination, i.e., anti-PD-L1 mAb, SN6j and SN6h (P<0.0001 between SN6j+anti-PD-L1 mAb and anti-PD-L1 mAb; P<0.0001 between SN6h+anti-PD-L1 mAb and anti-PD-L1 mAb; P<0.0001 between SN6j+anti-PD-L1 mAb and SN6j; P<0.0001 between SN6h+anti-PD-L1 mAb and SN6h). SN6h+anti-PD-L1 mAb show stronger antitumor activity than SN6j+anti-PD-L1 mAb (P<0.001). This result is consistent with above data in which GEMs bearing established 4T1 breast tumor were treated in a slightly different therapeutic protocol (see, FIG. 1).

FIG. 6 depicts prolongation of survival of GEMs bearing established 4T1 breast tumor by systemic administration of combination of anti-PD-L1 mAb (an immune checkpoint blocking antibody) and SN6h (an anti-ENG mAb) compared with administration of individual components of the combination. Panel a, Administration of SN6h+anti-PD-L1 mAb significantly prolonged survival of tumor-bearing mice compared with administration of anti-PD-L1 mAb only (P=0.0048). The median survival (survival of 50% of the mice) of the tumor-bearing mice was extended from 48 days (when treated with anti-PD-L1 mAb only) to 65 days (when treated with SN6h+anti-PD-L1 mAb). Panel b, Administration of SN6h+anti-PD-L1 mAb significantly prolonged survival of tumor-bearing mice compared with administration of SN6h only (P=0.0090). The median survival of the tumor-bearing mice was extended from 49 days (when treated with SN6h only) to 65 days (when treated with SN6h+anti-PD-L1 mAb).

FIG. 7 depicts suppression of growth of EMT6 breast tumor in novel GEMs expressing humanized ENG by systemic administration of i) CCL130 (an isotype-matched control mouse IgG, IgG1, of SN6j and SN6h)+rat IgG2b (an isotype-matched rat IgG, IgG2b, of anti-PD-L1 mAb), ii) CCL130+anti-PD-L1 mAb, iii) SN6j+anti-PD-L1 mAb, iv) SN6h+anti-PD-L1 mAb, or v) SN6j+SN6h+anti-PD-L1 mAb. Both SN6j and SN6h are anti-human ENG mAbs but define mutually non-overlapping distinctive epitopes. EMT6 murine breast cancer cells were inoculated s.c. into GEMs and mice with a similar size of established tumor were distributed nearly evenly into 5 groups of mice (n=10 for each group) at the onset of the therapy. CCL130, SN6j and SN6h were individually given i.v. at the dose of 100 μg/mouse and at 3-day intervals while rat IgG2b (from BioLegend) and anti-PD-L1 mAb (from clone 10F.9G2, BioLegend) were individually given i.v. at the dose of 175 μg/mouse and at 3-day intervals. The last injections of anti-PD-L1 mAb and rat IgG2b are indicated by an arrow on the abscissa while the last injections of CCL130, SN6j and SN6h are indicated by an arrow head on the abscissa. Statistically significant differences between different groups in the therapeutic efficacy are indicated by star symbols (*:P<0.05; ** : P<0.01; *** : P<0.001; **** : P<0.0001). CCL130+anti-PD-L1 mAb significantly suppressed tumor growth compared with CCL130+rat IgG2b (P<0.0001). SN6j+anti-PD-L1 mAb and SN6h+anti-PD-L1 mAb are more effective than CCL10+anti-PD-L1 mAb in the tumor suppression (P<0.0001 in each comparison). Addition of both SN6j and SN6h to anti-PD-L1 mAb strongly enhanced antitumor efficacy compared with SN6j+anti-PD-L1 mAb (P<0.001) and compared with SN6h+anti-PD-L1 mAb (P<0.0001).

### DETAILED DESCRIPTION

### Definitions

The term “angiogenesis-associated disease” is used herein to mean certain pathological processes in humans where angiogenesis is abnormally prolonged. Such angiogenesis-associated diseases include most types of human solid tumors, diabetic retinopathy, adult macular degeneration, chronic inflammatory diseases, rheumatoid arthritis, dermatitis; psoriasis, and stomach ulcers.

The term “angiogenesis inhibitor” is used herein to mean a biomolecule including, but not limited to, peptides, proteins, enzymes, polysaccharides, oligonucleotides, DNA, RNA, recombinant vectors, and drugs which function to inhibit angiogenesis. Angiogenesis inhibitors are known in the art and include natural and synthetic biomolecules such as paclitaxel, thrombospondin-1, thrombospondin-2, angiostatin, human chondrocyte-derived inhibitor of angiogenesis (“hCHIAMP”), cartilage-derived angiogenic inhibitor, platelet factor-4, gro-beta, human interferon-inducible protein 10 (“IP10”), interleukin 12, Ro 318220, tricyclodecan-9-yl xanthate (“D609”), irsogladine, medroxyprogesterone, a combination of heparin and cortisone, glucosidase inhibitors, genistein, thalidomide, diamino-antraquinone, herbimycin, ursolic acid, and oleanolic acid.

The term “antiangiogenic therapy” is used herein to mean therapy targeted to vasculature expressing endoglin (expressed at higher levels on proliferating vasculature as compared to quiescent vasculature); whether the therapy is directed against angiogenesis (i.e., the formation of new capillary blood vessels leading to neovascularization), and/or existing vasculature and relating to a disease condition (e.g., vascular targeting therapy).

The term “antibody fragment” or “fragment thereof” is used herein to mean a portion or fragment of an intact antibody molecule, wherein the fragment retains antigen-binding function; i.e., F(ab′)2, Fab′, Fab, Fv, single chain Fv (“scFv”), Fd′ and Fd fragments. Methods for producing the various fragments from Abs are well known to those skilled in the art (see, e.g., Pluckthurn, 1992, Immunol. Rev. 130:152-188).

The term “immunoconjugate” is used herein to mean a conjugate comprised of the anti-endoglin mAbs or a fragment thereof according to the present invention (or alternatively, an anti-endoglin mAb, or fragment thereof, that recognizes human vascular endothelial cells but lacks cross-reactivity with mouse endoglin) and at least one antitumor agent, or at least one angiogenesis inhibitor. Such antitumor agents are known in the art and include, but not limited to, toxins, drugs, enzymes, cytokines, radionuclides, photodynamic agents, and angiogenesis inhibitors. Toxins include ricin A chain, mutant Pseudomonas exotoxins, diphtheria toxoid, streptonigrin, saporin, gelonin, and pokeweed antiviral protein. Drugs include daunorubicin, methotrexate, and calicheamicins. Radionuclides include radiometals. Cytokines include transforming growth factor (TGF)-β, interleukins, interferons, and tumor necrosis factors. Photodynamic agents include porphyrins and their derivatives. The methods for complexing the anti-endoglin mAbs or a fragment thereof with at least one antitumor agent are well known to those skilled in the art (i.e., antibody conjugates as reviewed by Ghetie et al., 1994, Pharmacal. Ther. 63:209-34). Often such methods utilize one of several available heterobifunctional reagents used for coupling or linking molecules.

The term “immune checkpoint blocker” is used herein to mean a biomolecule including but not limited to, peptides, proteins, enzymes, polysaccharides, oligonucleotides, DNA, RNA, and drugs which function to unleash anti-tumor T cell responses. The immune checkpoint blocking antibody is a kind of immune checkpoint blocker and is widely used for immune checkpoint therapy of various cancers. The currently used immune checkpoint blocking antibodies include, but are not necessarily limited to, anti-CTLA-4 (cytotoxic T-lymphocyte-associated antigen 4) antibody, anti-PD-1 (programmed cell death protein-1) antibody, anti-PD-L1 (programmed cell death-ligand 1), antibody and anti-OX4 antibody.

The mechanisms of endoglin-targeted therapy are distinctively different from those of immune checkpoint blockade. Mechanisms of endoglin-targeted therapy involve anti-angiogenesis, apoptosis induction of microvascular endothelial cells, and modulation of TGF-beta-mediated and bone morphogenetic protein 9 (BMP9)-mediated signal transduction and down-regulation of Treg cells (She et al., Supra; Tsujie et al., Supra; Uneda et al., Supra; Seon et al., Supra; Karzai et al., Supra; Apolo et al., Supra), whereas mechanisms of immune checkpoint blockade therapy involve blocking of the pathways inhibiting the endogenous immune response to cancer in the tumor-bearing host (Sharma and Allison, Supra). Importantly, adverse events of ENG-targeted therapy in cancer patients do not overlap with those of immune checkpoint blockade in cancer patients. Adverse events of anti-endoglin mAb TRC105 when it was administered as a single agent into cancer patients included infusion-related reaction, anemia, fatigue, telangiectasia and nausea (Rosen et al., Supra; Karzai et al., Supra; Apolo et al., Supra). Adverse events of anti-CTLA-4 mAb, anti-PD-1 mAb and anti-PD-L1 mAb included asthenia, rash, adverse cutaneous reactions, decreased appetite, diarrhea and nausea (Cousin and Italiano, Supra; Simone et al., Supra). We tested whether the combination of ENG-targeted therapy and checkpoint blockade will exert additive or synergistic antitumor efficacy, and to determine if toxicity of the combination will substantially increase compared with known toxicity of each component of the combination.

Without intending to be constrained by any particular theory, the rationale for these tests is as follows: T cell immunity plays an important role in the endoglin-targeted therapy as indicated by our comparative studies between immunocompetent and immunodeficient mice (Tsujie et al., Supra). This involvement of T cell immunity is consistent with a recent finding that the level of regulatory T cells (Treg cells) among CD4⁺ T cells significantly decreased after treatment with TRC105 (used as a single agent) compared with baseline (n=7, P=0.016 for urothelial cancer; n=19, P=0.032 for Cycle 1 Day 15 for prostate cancer) in a phase II clinical trial of TRC105 in adults with advanced/metastatic urothelial carcinoma (Karzai et al., Supra) and in a phase I clinical trial of TRC105 in patients with advanced/metastatic prostate cancer. This finding is interesting in view of the fact that TGF-beta plays an important role in Treg cell development (Campbell, 2015, J. Immunol., 195: 2507-2513; Plitas and Rudensky, 2016, Cancer Immunol. Res., 4: 721-725) and endoglin is a co-receptor of TGF-beta (Cheifetz et al., 1992, J. Biol. Chem., 267: 19027-19030). Importantly Treg cells in the tumor microenvironment present a major impediment to effective immunotherapy of cancer.

The term “isotype control immunoglobulin” is used herein-to mean a species specific (e.g. raised in the same species as the antibody to which it is compared), isotype-matched (e.g., of the same immunoglobulin (Ig) class and subclass as the antibody to which it is compared) Ig which does not bind with specificity to the antigen to which the compared antibody has binding specificity, as will be more apparent from the following embodiments.

The term “mammalian host” or “host” is used herein to mean a mouse or a human.

The term “monoclonal antibody (mAb)”, as denoted as having binding specificity for an epitope of endoglin, is used herein-to mean murine monoclonal antibodies and engineered (e.g., recombinant)/antibody molecules made therefrom in which the binding specificity for an epitope of endoglin and includes chimeric or “humanized” antibodies, as will be more apparent from the following embodiments.

The term “tumor” is used herein to mean a tumor expressing endoglin at moderate to high levels (as compared to expression by normal tissue of the same type) such as human leukemias, including non-T-cell-type (non-T) acute lymphoblastic leukemia (ALL), myelo-monocytic leukemia; and human solid tumors, with its surrounding vasculature expressing endoglin at moderate to high levels (as compared to expression by normal tissue of the same type) including angiosarcoma, breast carcinoma, cecum carcinoma, colon carcinoma, lung carcinoma, melanoma, osteosarcoma, ovarian carcinoma, parotid tumor, pharyngeal carcinoma, prostate carcinoma, and rectosigmoid carcinoma.

A drawback to conventional chemotherapy and radiotherapy is the lack of selectively delivering the therapy to its intended target, diseased tissue, rather than to normal tissue. Monoclonal antibodies have been used to deliver therapeutics with greater target specificity, thereby reducing toxicity. Murine Abs or fragments thereof have been used to treat human disease, often with modest to substantial clinical efficacy (see, e.g., Ghetie et al., 1994, supra). Studies show that human anti-mouse antibody immune response will develop after murine mAbs are injected into patients repeatedly. Therefore, murine mAbs are chimerized or humanized before they are repeatedly injected into patients for therapeutic purposes (Carter, 2006, Nature Rev. Immunol., 6: 343-357).

The present invention utilizes GEMs stably expressing novel human/mouse chimeric (humanized) endoglin which are described in U.S. Pat. No. 9,315,582, from which the description of transgenic mice is incorporated herein by reference. A major impediment in evaluating therapeutic potential of anti-human endoglin (hENG) mAbs is that most anti-hENG mAbs show little cross-reactivity with mouse/rat ENG or endothelial cells. This lack of cross-reactivity hampers animal model studies of anti-hENG mAbs that are necessary before these mAbs are tested in cancer patients. To overcome this obstacle, we developed GEMs that stably express humanized endoglin (Toi et al., Supra) It should be noted that mice lacking ENG die from defective vascular development (Li et al., 1999, Science 284: 1534-1537) and mutations of hENG in human are associated with hereditary hemorrhagic telangiectasia type 1 (HHT 1; McAllister et al., 1994, Nature Genetics 8: 345-351). Fortunately we found that our GEMs develop normally and do not show any signs of hemorrhagic telangiectasia-like symptoms. The results indicate that the generated humanized chimeric ENGs are physiologically functional in mice. Immunohistochemical (IHC) analysis of tumor tissues from the GEMs showed that microvessels of tumors (colon26 and 4T1) of GEMs reacted strongly with 7 anti-hENG mAbs (SN6, SN6c, SN6d, SN6h, SN6i and SN6j) and weakly with 1 anti-hENG mAb (SN6g) (Toi et al, Supra).

The following Examples are intended to illustrate but not limit embodiments of the invention.

### Example 1

Production of Anti-Human Endoglin Monoclonal Antibodies (Anti-hENG mAbs)

Anti-hENG mAb SN6 was generated by immunizing a mouse with a tumor-associated antigen-enriched fraction of cell membrane glycoproteins from ENG-expressing human leukemia cells (Haruta and Seon, 1986, Proc. Natl. Acad. Sci. USA 83: 7898-7902). The two anti-hENG mAbs, termed SN6j (from hybridoma clone Y4-2F1) and SN6h (from hybridoma clone G4-2C2), of the present invention were generated by immunization of mice with a purified hENG preparation (Matsuno et al., 1999, Clin. Cancer Res., 5:371-382; Takahashi et al., 2001, Clin. Cancer Res., 7:524-532). hENG was purified from human acute lymphoblastic leukemia (ALL) cells. Cell membrane glycoproteins were isolated using detergent extraction and lectin affinity chromatography as described previously (Haruta and Seon, Supra, herein incorporated by reference). The isolated glycoproteins were applied to an immunoaffinity column containing anti-endoglin mAb SN6 (Haruta and Sean, 1986, supra) which had been equilibrated with 25 mM Tris-HCl, pH 8.0 containing 0.5% taurocholate, 0.15 M NaCl, 2 mM EDTA, 0.03% NaN3 and 0.5 mM phenylmethylsulfonyl fluoride. The bound materials were eluted with 50 mM diethylamine-HCl, pH 11.3 containing 0.5% taurocholate, 2 mM EDTA, 0.03% NaN3 (“alkaline buffer”). The eluate was immediately neutralized by the addition of one-tenth volume of 0.5 M Tris-HCl buffer, pH 7.1. The eluted material was reapplied to the immunoaffinity column and the bound material was eluted with the alkaline buffer further containing 0.01% cytochrome-c (a 12.4 kD carrier protein) and neutralized. The eluted material was dialyzed and concentrated using ultrafiltration (e.g., with a YM-10 membrane}. This purification process was carried out at 4-6° C. Purification of hENG was monitored by a solid phase radioimmunoassay using mAb SN6, and confirmed by gel electrophoresis with silver staining. The resultant hENG preparation contained a single, major component of 170 kD under unreduced conditions, and 92 kD under reduced conditions.

In a first immunization protocol, 2 female BALB/c mice were immunized with the isolated hENG following an immunization protocol described previously (Seon et al., 1983, Proc. Natl. Acad. Sci. USA, 80:845-849) with modifications. Briefly, an antigen solution comprising 10 μg of the hENG preparation in 100 μl of 10 mM Tris-HCl buffer, pH 7.5, with 0.5% taurocholate, 0.15 M NaCl, and 14 μg cytochrome-c, was mixed with an equal volume of adjuvant (e.g., Fruend's complete) and then injected subcutaneously at multiple sites on each of the mice. In addition, 1×109 Bordetella pertussis bacteria in 100 μl saline were injected at different sites. Two booster immunizations of the antigen solution in adjuvant were administered subcutaneously. A last immunization comprising 40 μl antigen solution containing 8 μg hENG preparation mixed with 200 μl saline was administered intraperitoneally. The spleens were removed and fused with P3/NS1/1-Ag4-1 (NS-1) mouse myeloma cell line 4 days after the last immunization. Cell fusion, hybridoma screening, and immunoglobulin class determination were performed as described previously (Haruta and Seon, 1986, supra). In a second immunization protocol, a female BALB/c mouse was immunized with the isolated hENG as described for the first experiment, but without the administration of B. pertussis. Eleven hybridomas that include hybridoma Y4-2F1 and hybridoma G4-2C2 were generated by these immunizations and they produce individually different anti-hENG mAbs that were further characterized.

Seven of the eleven mAbs generated using these immunization protocols demonstrated binding specificity for the hENG part of the human/mouse chimeric ENG that is stably expressed in the knockin mice; these seven mAbs included SN6j and SN6h (Toi et al., 2015, Supra). The anti-endoglin antibody SN6j is produced by a hybridoma designated by American Type Culture Collection Deposit No. HB-12171, and is described in U.S. Pat. No. 7,691,374, the description from which is incorporated herein by reference. The hybridoma which produces the antibody referred to herein as SN6h has not been deposited with the American Type Culture Collection.

### Example 2

Potentiation of Antitumor Efficacy by Combination of an Anti-Endoglin (ENG) Antibody with an Immune Checkpoint Blockade Antibody

This Example illustrates a method for combination therapy that combines antiangiogenic therapy and immune checkpoint blockade therapy. Antiangiogenic therapy includes therapies against tumor vasculature expressing ENG or against excessive vascularization present in other angiogenesis-associated diseases.

While an anti-hENG) mAb may be administered by routes other than intravenously (i.v.), a preferred embodiment of the illustration is i.v. administration of the mAb. This is because it is primarily the proliferating vasculature comprising the angiogenesis that is the target of the therapy. Thus, administering the mAb i.v. saturates the targeted vasculature much quicker than if another route of administration is used. Additionally, the intravenous route allows for the possibility of further targeting to specific tissues. In a variation of this embodiment, a catheter may be used to direct the mAb to the location of the target angiogenesis. For example, if tumor angiogenesis is the target of the anti-angiogenic therapy, and if the tumor is located in the liver, then the mAb may be delivered into the hepatic portal vein using a catheter. In this variation, there is less systemic distribution of the mAb, thereby further minimizing any potential side effects from antiangiogenic therapy.

Anti-hENG mAbs SN6j and SN6h were used to illustrate the antiangiogenic therapy according to the method of the present invention.

Immune checkpoint blockade therapy utilizes strategies to unleash T cells against tumors and can be applied to a wide variety of tumors (Sharma and Allison, 2015, Cell 16:205-214). Immune checkpoint blockade therapy may result in durable responses but only in a fraction of patients (Sharma and Allison, 2015, Supra). Therefore, immune checkpoint blockade therapy needs to be combined with other therapy. In the present invention, ENG-targeted therapy is combined with the immune checkpoint blockade therapy. Many clinical trials by a number of investigators indicated that anti-PD-1 mAb and anti-PD-L1 mAb may be the most effective anticancer agents among the known immune checkpoint blockade agents (e.g., Topalian et al., 2012, New Engl. J. Med. 366:2443-2454; Brahma et al., Supra; Moreno and Ribas, 2015, British J. Cancer 112:1421-1427; Boussiotis, 2016, New Engl. J. Med. 375:1767-1778). These clinical trials indicate that anti-PD-1 mAb and anti-PD-L1 mAb may be superior to anti-CTLA-4 mAb with regard to the prolonged progression-free survival and less high-grade toxicity (Schachter et al., 2015, New Engl. J. Med. 372:2521-2532). Therefore we selected anti-PD-L1 mAb as the immune checkpoint blockade agent in the studies of this patent application. Anti-PD-L1 mAb is designed to inhibit the interaction of PD-L1 with PD-1 and B7.1, relieving the inhibition of T-cell activity and allowing for antitumor immune response.

The combination significantly potentiated antitumor efficacy compared with either immune checkpoint blockade therapy or ENG-targeted therapy.

Anti-PD-L1 mAb was used to illustrate the immune checkpoint blockade antibodies.

Seven sets of illustrative therapeutic protocols were used, as follows.

### Example 3

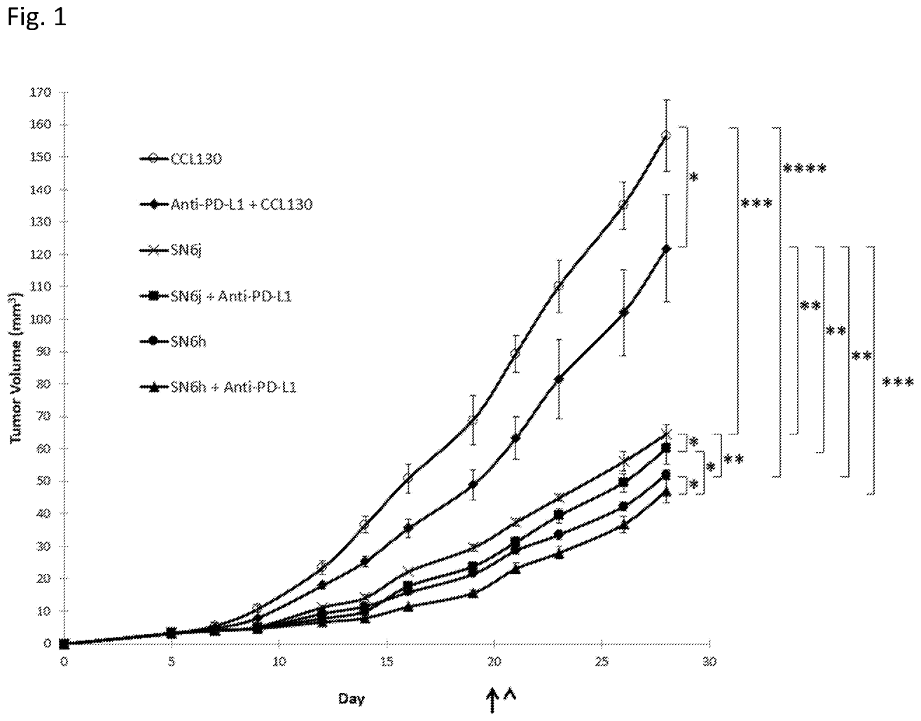

In the first protocol, 4T1 murine breast cancer cells were inoculated subcutaneously (s.c.) into the flank of GEMs stably expressing human/mouse chimeric ENG. Mice bearing palpable established tumors of a similar size were distributed evenly into six groups (n=7 for each group) at the onset of the therapy. Individual mice with established tumors in each of 3 groups of tumor-bearing mice were treated by intravenous (i.v.) administration of 200 μg/mouse of SN6j, SN6h or isotype-matched control IgG (CCL130) via the tail vein at 2 days intervals. Each of the remaining 3 groups of tumor-bearing mice was treated by i.v. administration of anti-PD-L1 mAb plus CCL130, anti-PD-L1 mAb plus SN6j or anti-PD-L1 mAb plus SN6h. The PD-L1 mAb was administered at the dose of 300 μg/mouse and at 3 days intervals. Mice were followed daily, and tumor size and body weight were measured at 2 or 3 days interval (shown in FIG. 1). Anti-PD-L1 mAb shows a weak but significant tumor suppressive activity compared with the control, CCL130 (p<0.05). Both anti-ENG mAb SN6j and SN6h strongly suppressed tumor growth compared with CCL130 ((p<0.001 and <0.0001, respectively). Addition of anti-PD-L1 mAb to SN6j or SN6h strongly potentiates antitumor activity compared with anti-PD-L1 mAb+CCL130 (p<0.01 for anti-PD-L1 mAb+SN6j and p<0.001 for anti-PD-L1 mAb+SN6h). In addition, the addition of anti-PD-L1 mAb potentiates antitumor activity compared with SN6j alone (p<0.05) and SN6h alone (p<0.05). SN6h is more effective than SN6j for tumor suppression (p<0.01).

### Example 4

In the second protocol, metastasis suppressive activities of anti-PD-L1 mAb, anti-ENG mAb and combination of anti-PD-L1 mAb with anti-ENG mAb were evaluated. 4T1 breast tumor that were formed by subcutaneous (s.c.) injection of the tumor cells metastasizes spontaneously into distant organs including lung. Six groups (n=7 for each group of mice) of GEMs expressing novel humanized ENG were inoculated s.c. with 4T1 breast cancer cells and treated as described in FIG. 1. Mice were sacrificed 7 days after the last treatment, and metastatic nodules in the lungs were counted. Addition of anti-ENG mAb SN6j or SN6h to an immune checkpoint blocking anti-PD-L1 mAb potentiates metastasis suppressive activity compared with anti-PD-L1 mAb+CCL130 (p=0.0000099 for SN6j and p=0.0000015 for SN6h). It is remarkable that metastasis is completely suppressed in the lungs of 3 of 7 mice that received SN6h plus anti-PD-L1 mAb. SN6j and SN6h are also effective for suppressing metastasis compared with control IgG CCL130 (p=0.000067 for SN6j and p=0.0000032 for SN6h).

### Example 5

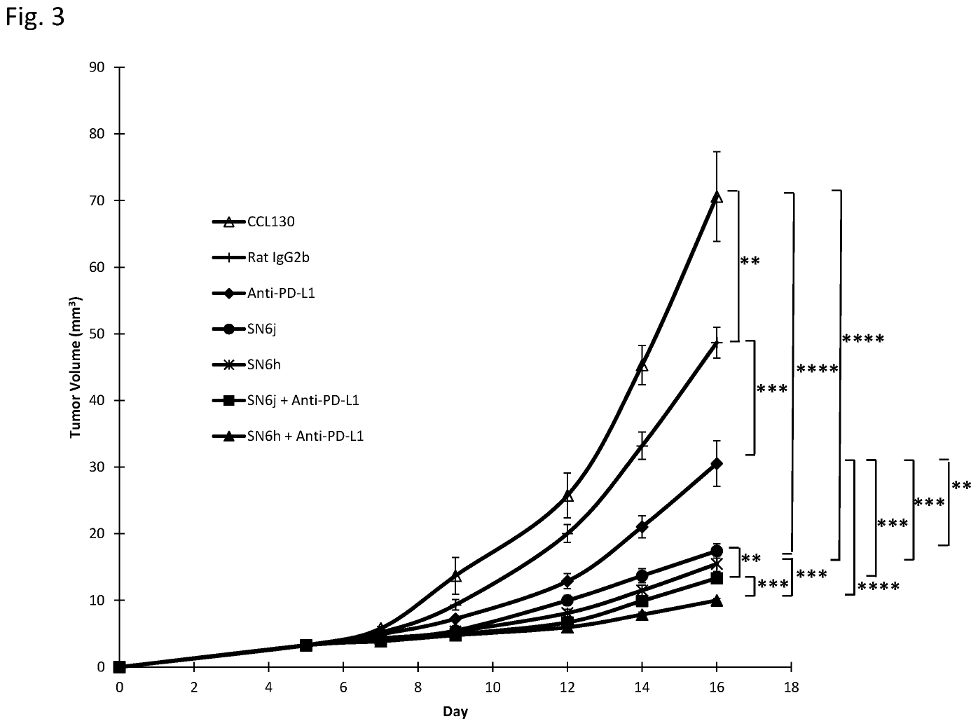

In the third protocol, Colon 26 colorectal tumor cells were subcutaneously (s.c.) injected into GEMs expressing humanized endoglin (ENG) as described in FIG. 3. Mice with a similar size of tumors were distributed nearly evenly into 7 groups (n=8 for each of 5 therapy groups and n=7 for each of 2 control groups) at the onset of the therapy. Individual groups of mice with established tumors were treated by intravenous (i.v.) administration of an isotype-matched control mouse IgG CCL130 (IgG1), an isotype-matched control rat IgG (IgG2b from BioLegend), rat anti-mouse PD-L1 mAb (IgG2b from clone 10F.9G2, BioLegend), mouse anti-human ENG mAb SN6j (IgG1) and SN6h (IgG1) as described in FIG. 3. The remaining two groups of mice were treated by i.v. administration of SN6j+anti-PD-L1 mAb and SN6h+anti-PD-L1 mAb, respectively as described in FIG. 3. CCL130, SN6j and SN6h were individually administered at the dose of 150 μg/mouse and at 3-day intervals, whereas rat control IgG2b and anti-PD-L1 mAb were individually given at the dose of 300 μg/mouse and at 3-day intervals. Rat control IgG2b suppresses tumor growth significantly in the tumor-bearing mice compared with CCL130 mouse control IgG (p<0.01). This tumor suppressive activity of rat IgG2b in tumor-bearing mice is probably caused by side effects of the immune response of mice against the hetero-species rat IgG. Rat anti-mouse PD-L1 mAb shows significant tumor suppressive activity compared with the isotype-matched control rat IgG2b (p<0.001). SN6j and SN6h show stronger antitumor activity (p<0.0001, respectively, compared with CCL130). Combination of the anti-PD-L1 mAb with either SN6j or SN6h potentiates antitumor activity compared with either anti-PD-L1 mAb (p<0.001 and p<0.0001, respectively, for SN6j and SN6h) or with anti-ENG mAb (p<0.01 and p<0.001, respectively, for SN6j and SN6h). SN6h+anti-PD-L1 mAb is more potent than SN6j+anti-PD-L1 in the tumor growth suppression (p<0.001). Statistically significant differences between different groups in the tumor suppression are indicated by star symbols (* : <0.05; ** : <0.01; *** : <0.001; **** : <0.0001).

### Example 6

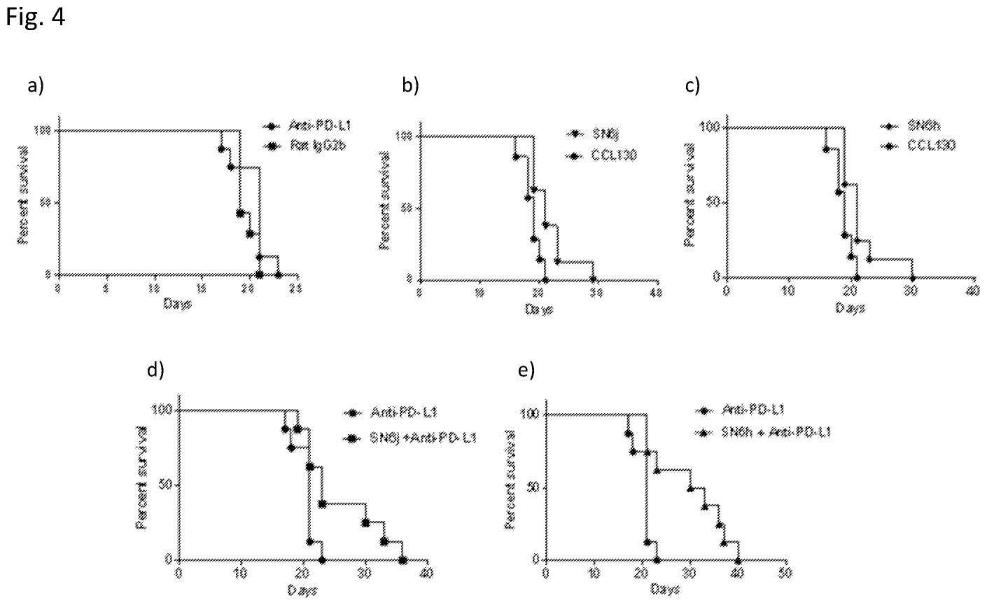

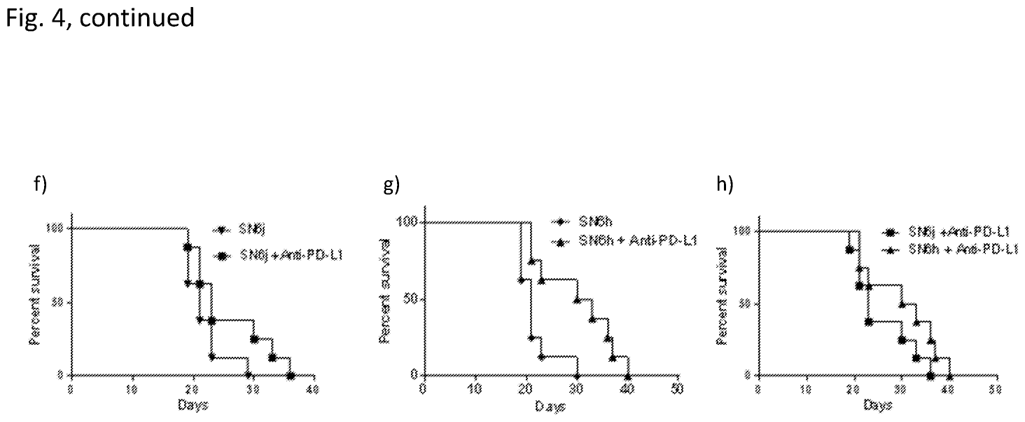

In the fourth protocol, prolongation of survival of tumor-bearing mice was measured after systemic administration of ENG-targeting antibody or combination of an immune checkpoint blockade antibody with an ENG-targeting antibody. Colon 26 colorectal tumor cells were inoculated s.c. into GEMs expressing humanized ENG and 7 groups of the tumor-bearing mice (n=8 for each of 5 therapy groups and n=7 for each of two control groups) were treated as described in Figure. 3. In the present test, survival of each group of mice was monitored and survival of relevant pairs of groups of mice is presented in FIG. 4 panel a) to panel h). FIG. 4 panel a) Anti-PD-L1 mAb shows a weak trend to extension of survival of tumor-bearing mice compared with the isotype-matched control rat IgG (IgG2b), but the extension is not statistically significant (p=0.163). FIG. 4, panel b) Anti-ENG mAb SN6j is effective for extending the survival of the tumor bearing mice compared with the isotype-matched control mouse IgG1, CCL130 (p=0.0182). FIG. 4, panel c) Another anti-ENG mAb SN6h is also effective for extending the survival of the tumor-bearing mice compared with CCL130 (p=0.0240). FIG. 4, panel d) SN6j+anti-PD-L1 mAb is effective for prolonging the survival of tumor-bearing mice compared with anti-PD-L1 mAb alone (p=0.0353). FIG. 4, panel e) SN6h+anti-PD-L1 mAb is highly effective for prolonging the survival of the tumor-bearing mice compared with anti-PD-L1 mAb alone (p=0.0033). FIG. 4, panel f) Addition of anti-PD-L1 mAb to SN6j is not sufficiently effective for extending the survival compared with SN6j alone (p=0.0847). FIG. 4, panel g) Addition of anti-PD-L1 mAb to SN6h strongly extended the survival of the tumor-bearing mice compared with SN6h alone (p=0.0078). FIG. 4, panel h) SN6h+anti-PD-L1 mAb appears to be more effective than SN6j+anti-PD-L1 mAb in treating the tumor-bearing mice but the difference in the survival extension between the two groups is not statistically significant (p=0.147). Nevertheless, SN6h+anti-PD-L1 mAb is more effective for survival prolongation of the tumor-bearing mice than either of anti-PD-L1 mAb alone (p=0.0033) and SN6h alone (p=0.0078). The statistical analysis of the survival was performed by use of Log-rank (Mantel-Cox) test and Gehan-Breslow-Wilcoxon test. The p values obtained by the two tests are similar. The presented p values are those that were obtained by the Log-rank (Mantel-Cox) test. Analysis of the survival extension by the combination therapy shows that the combination of SN6h and anti-PD-L1 mAb exerts synergistic effect on the survival extension of the tumor-bearing mice compared with the individual components of the combination as described in the legend to FIG. 4.

### Example 7

In the fifth protocol, 4T1 murine breast tumor cells were inoculated s.c. into GEMs and mice with a similar size of established tumor were distributed nearly evenly into 7 groups of mice (n=10 for each group) at the onset of the therapy. The rat anti-mouse PD-L1 mAb 10F.9G2 (from BioLegend) or the control rat IgG2b (from BioLegend) was given intravenously (i.v.) at the dose of 250 μg/mouse and at 3-day intervals. SN6j, SN6h or the control IgG CCL130 was given i.v. at the dose of 200 μg/mouse at 2-day intervals. The last injection of anti-PD-L1 mAb or rat IgG2b is indicated by an arrow on the abscissa while the last injection of CCL130, SN6j or SN6h is indicated by an arrow head. Statistically significant differences between different groups in the therapeutic efficacy are indicated by star symbols (* :P<0.05; ** : P<0.01; *** : P<0.001; **** : P<0.0001). Mice were followed daily, and tumor size and body weight were measured at 2 or 3 days intervals. Rat IgG2b shows significant activity against tumor growth compared with CCL130, the mouse control IgG (P<0.001); this activity of rat IgG2b in mice is probably caused by side effects of the immune response of mice against hetero-species rat IgG. Rat anti-mouse PD-L1 mAb shows strong tumor-suppressive activity compared with the isotype-matched control rat IgG2b (P<0.0001). Both anti-ENG mAb SN6j and mAb SN6h show stronger tumor suppressive activities than anti-PD-L1 mAb (P<0.001 and <0.0001, respectively). Combination of SN6j or SN6h with anti-PD-L1 mAb strongly enhanced antitumor activity compared with individual components of the combination, i.e., anti-PD-L1 mAb, SN6j and SN6h (P<0.0001 between SN6j+anti-PD-L1 mAb and anti-PD-L1 mAb; P<0.0001 between SN6h+anti-PD-L1 mAb and anti-PD-L1 mAb; P<0.0001 between SN6j+anti-PD-L1 mAb and SN6j; P<0.0001 between SN6h+anti-PD-L1 mAb and SN6h). SN6h+anti-PD-L1 mAb show stronger antitumor activity than SN6j+anti-PD-L1 mAb (P<0.001). This result is consistent with that of the earlier experiment in which GEMs bearing established 4T1 breast tumor was treated in a slightly different therapeutic protocol as presented in FIG. 1.

### Example 8

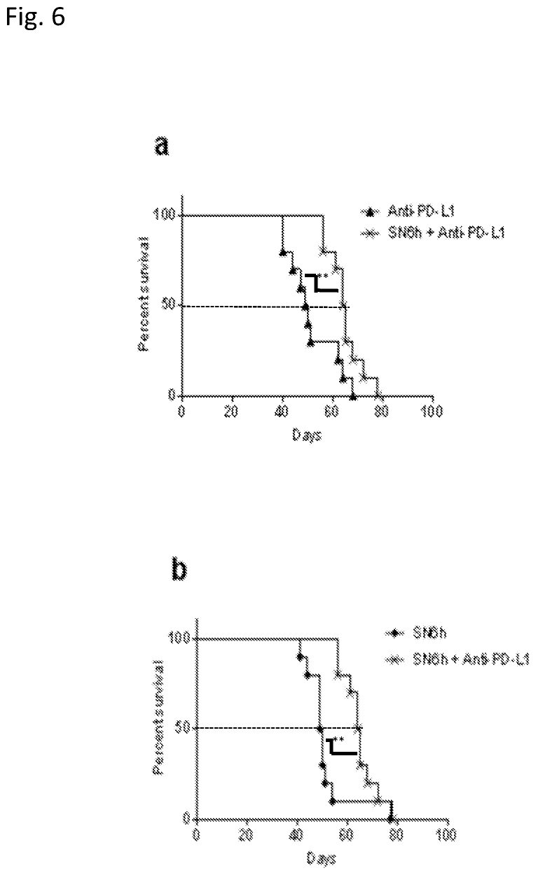

In the sixth protocol, prolongation of survival of GEMs bearing established 4T1 breast tumor was compared between anti-PD-L1 mAb and SN6h+anti-PD-L1 mAb (in FIG. 6, panel a) and between SN6h and SN6h+anti-PD-L1 mAb (in FIG. 6, panel b). 4T1 breast cancer cells were inoculated s.c. into GEMs as described in the protocol of the fifth set of the protocol. Anti-PD-L1 mAb and SN6h were administered i.v. into the tumor-bearing mice as described in the fifth protocol. For this embodiment, FIG. 6, panel a: shows administration of SN6h+anti-PD-L1 mAb significantly prolonged survival of tumor-bearing mice compared with administration of anti-PD-L1 mAb only (P=0.0048). The median survival (survival of 50% of the mice) of the tumor-bearing mice was extended from 48 days (when treated with anti-PD-L1 mAb only) to 65 days (when treated with SN6h+anti-PD-L1 mAb). FIG. 6, panel b shows that administration of SN6h+anti-PD-L1 mAb significantly prolonged survival of tumor-bearing mice compared with administration of SN6h only (P=0.0090). The median survival of the tumor-bearing mice was extended from 49 days (when treated with SN6h only) to 65 days (when treated with SN6h+anti-PD-L1 mAb).

### Example 9

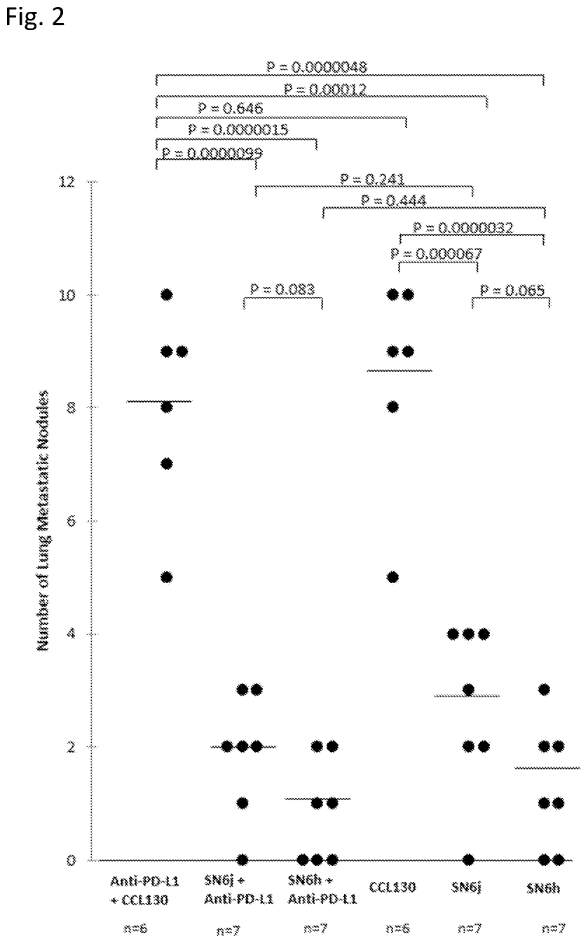

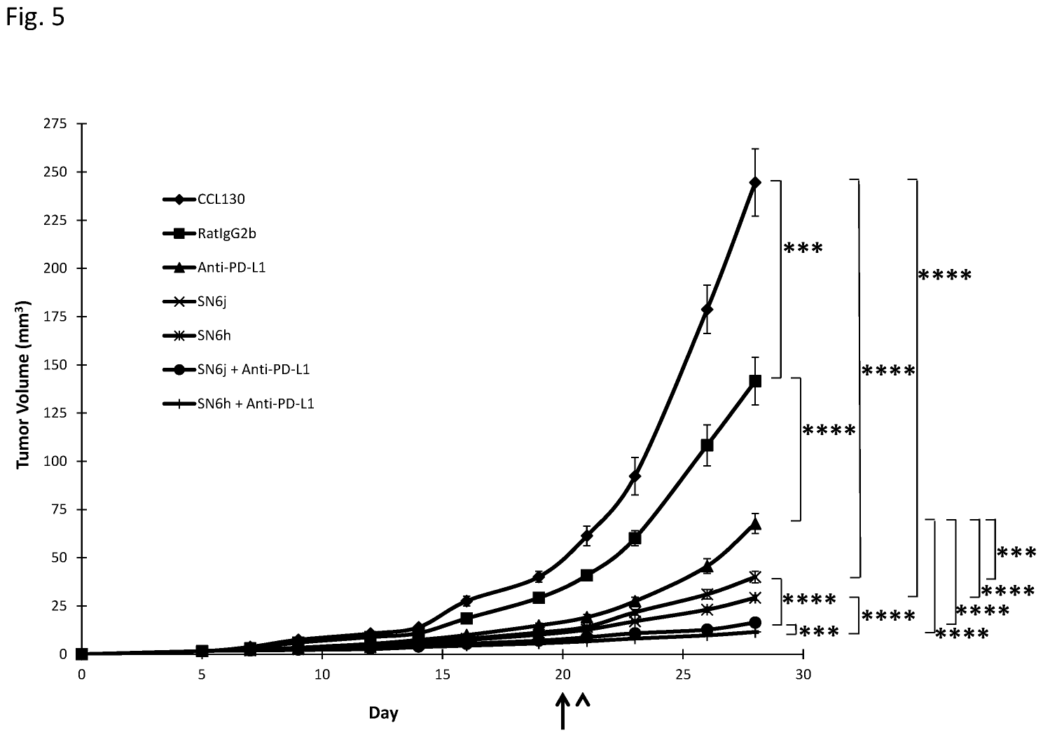

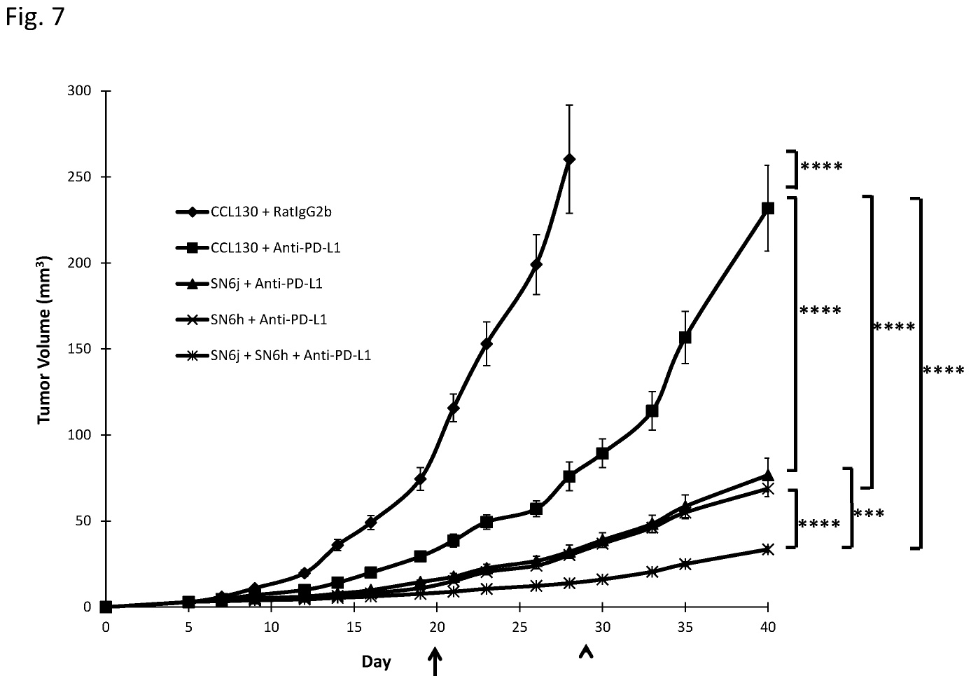

In the seventh protocol, as illustrated by the results summarized in FIG. 7, EMT6 murine breast cancer cells were inoculated s.c. into GEMs and mice with a similar size of established tumor were distributed nearly evenly into 5 groups of mice (n=10 for each group) at the onset of the therapy. CCL130, SN6j and SN6h were individually given i.v. at the dose of 100 μg/mouse and at 3-day intervals while rat IgG2b (from BioLegend) and anti-PD-L1 mAb (from clone 10F.9G2, BioLegend) were individually given i.v. at the dose of 175 μg/mouse and at 3-day intervals. The last injections of anti-PD-L1 mAb and rat IgG2b are indicated by an arrow on the abscissa while the last injections of CCL130, SN6j and SN6h are indicated by an arrow head on the abscissa. Statistically significant differences between different groups in the therapeutic efficacy are indicated by star symbols (* :P<0.05; ** : P<0.01; *** : P<0.001; **** : P<0.0001). Mice were followed daily, and tumor size and body weight were measured at 2 or 3 days intervals. CCL130+anti-PD-L1 mAb significantly suppressed tumor growth compared with CCL130+rat IgG2b (P<0.0001). SN6j+anti-PD-L1 mAb and SN6h+anti-PD-L1 mAb are more effective than CCL130+anti-PD-L1 mAb in the tumor suppression (P<0.0001 in each comparison). Addition of both SN6j and SN6h to anti-PD-L1 mAb strongly enhanced antitumor efficacy compared with SN6j+anti-PD-L1 mAb (P<0.001) and compared with SN6h+anti-PD-L1 mAb (P<0.0001). Results of this therapeutic experiment of GEMs bearing EMT6 breast tumor are consistent with those of experiments of GEMs bearing 4T1 breast tumor (FIGS. 1, 2, 5 and 6) and Colon 26 colorectal tumor (FIGS. 3 and 4). Results demonstrate that addition of anti-ENG mAb SN6h and anti-ENG mAb SN6j to anti-PD-L1 mAb strongly potentiates antitumor efficacy.

While the invention has been described through specific embodiments, routine modifications will be apparent to those skilled in the art and such modifications are intended to be within the scope of the present invention.

1. A method comprising administering to a human individual in need thereof: i) an immune checkpoint inhibitor; and 2) a monoclonal antibody (mAb) or antigen binding fragment thereof, wherein the mAb or the fragment thereof binds with specificity to human endoglin.

2. The method of claim 1, wherein the immune checkpoint inhibitor comprises an anti-programmed cell death protein-1 (anti-PD-1) antibody or PD-1 binding fragment thereof, or a programmed cell death-ligand 1 (anti-PD-L1) or a PD-L1 binding fragment thereof.

3. The method of claim 2, wherein the immune checkpoint inhibitor comprises the anti-PD-L1 antibody or the PD-L1 binding fragment thereof.

4. The method of claim 1, wherein the mAb that binds to the human endoglin is a chimeric or humanized antibody.

5. The method of claim 4, wherein the individual has cancer, and wherein administering a combination of the immune checkpoint inhibitor and the anti-human endoglin mAb that binds to the human endoglin results in improvement of survival time of the individual relative to survival time from administering the immune checkpoint inhibitor or the mAb alone.

6. The method of claim 4, wherein the mAb that binds to the human endoglin comprises SN6h or SN6j.

7. The method of claim 5, wherein the mAb that binds to the human endoglin comprises SN6h or SN6j.

8. The method of claim 4, wherein the administering a combination of the immune checkpoint inhibitor and the mAb that binds to the human endoglin results in an inhibition of growth of a tumor and/or a reduction in tumor volume in the individual.

9. The method of claim 8, wherein the mAb that binds to the human endoglin comprises SN6h or SN6j.

10. The method of claim 4, wherein the administering a combination of the immune checkpoint inhibitor and the mAb that binds to the human endoglin results in an inhibition of metastasis from a primary tumor in the individual.

11. The method of claim 5, wherein the administering a combination of the immune checkpoint inhibitor and the mAb that binds to the human endoglin results in an inhibition of metastasis from a primary tumor in the individual.

12. The method of claim 6, wherein the administering a combination of the immune checkpoint inhibitor and the mAb that binds to the human endoglin results in an inhibition of metastasis from a primary tumor in the individual.

13. The method of claim 1, wherein the fragment of the mAb that binds to the human endoglin is selected from the group consisting of Fab, Fab′, (Fab′)₂, Fv, or a single chain (ScFv).

14. The method of claim 13, wherein the fragment of the mAb that binds to the human endoglin is from SN6h or SN6j.

15. The method of claim 1, wherein the individual is determined to have a cancer that is resistant to an immune checkpoint inhibitor prior to the administering the combination of the immune checkpoint inhibitor and the mAb or the fragment thereof that binds to the human endoglin, and wherein administering the combination potentiates the efficacy of the immune checkpoint inhibitor in the individual.

16. The method of claim 15, wherein the mAb that binds to the human endoglin is a chimeric or humanized antibody.

17. The method of claim 16, wherein administering a combination of the immune checkpoint inhibitor and the anti-human endoglin mAb that binds to the human endoglin results in improvement of survival time of the individual relative to survival time from administering the immune checkpoint inhibitor or the mAb alone.

18. The method of claim 16, wherein the mAb that binds to the human endoglin comprises SN6h or SN6j.

19. The method of claim 17, wherein the mAb that binds to the human endoglin comprises SN6h or SN6j.

20. The method of claim 15, wherein the fragment of the mAb that binds to the human endoglin is selected from the group consisting of Fab, Fab′, (Fab′)₂, Fv, or a single chain (ScFv).

21. The method of claim 20, wherein the fragment is from SN6h or SN6j.
